(19) United States
(12) Patent Application Publication
(10) Pub. No.: US 2020/0327957 A1
(43) Pub. Date: Oct. 15, 2020

(54) DETECTION OF DELETIONS AND COPY NUMBER VARIATIONS IN DNA SEQUENCES

(71) Applicant: ANCESTRY.COM DNA, LLC, Lehi, UT (US)

(72) Inventors: Velina KOZAREVA, Cambridge, MA (US); Nigel DELANEY, Davis, CA (US)

(21) Appl. No.: 16/772,739

(22) PCT Filed: Dec. 12, 2018
(86) PCT No.: PCT/US2018/065241

§ 371 Date: Jun. 12, 2020

Related U.S. Application Data
(60) Provisional application No. 62/598,873, filed on Dec. 14, 2017.
(60) Provisional application No. 62/598,783, filed on Dec. 14, 2017.

(51) Int. Cl. G16B 20/20; G16B 30/10; G16B 20/10

(52) U.S. Cl. CPC G16B 20/20; G16B 20/10; G16B 30/10

(57) ABSTRACT

Methods and systems are provided for improved detection of a relatively large predefined deletion using short read exome sequencing. Short read exome sequences of continuous exomes segments of a genome may be obtained each having a length of base pairs that is less than or equal to a threshold value. A target sequence of a reference genome may be stored that has a predefined deletion of a reference sequence having a length of base pairs that is relatively larger than the threshold value, such that a segment positioned after the deletion is shifted to abut a segment positioned prior to the deletion. Instances of short read exome sequences may be detected that straddle both the segment positioned after the deletion and the segment positioned prior to the deletion, wherein both segments falling within the relatively shorter length of the short read exome sequences indicates that the deletion has occurred.

### CROSS-REFERENCE TO RELATED APPLICATIONS

This application claims priority to U.S. Provisional Patent Application No. 62/598,873, filed Dec. 14, 2017, and to U.S. Provisional Patent Application No. 62/598,783, filed Dec. 14, 2017, both of which are incorporated herein by reference in their entirety.

### FIELD

Embodiments relate to identifying copy number variants (CNVs) and detecting deletion of a reference genetic sequence for screening for genetic disease. Example genetic diseases caused by CNVs include, but are not limited to, Duchenne muscular dystrophy (DMD) and Becker muscular dystrophy.

### BACKGROUND

Structural variation of the genome is the variation of an organism's chromosome, which is made up of DNA. A genomic structural variation may affect nucleic acid sequence length of from for example approximately 1 Kb to 3 Mb. One type of structural genomic variation is a copy number variant (CNV) in which the DNA sequence of a gene varies in copy-number, e.g., is duplicated or deleted. Copy number variation occurs in part or all of a gene or in a genomic segment containing several genes. Certain CNVs are associated with or cause genetic diseases.

In recent years, analysis for copy number variants (CNVs), which have been demonstrated to be causal in a number of genetic disorders, has become a prominent component of clinical testing for diagnosis and prenatal screening. However, while the vast majority of CNV analysis is performed using targeted microarray technologies, many clinical tests rely predominantly on high-throughput sequencing in order to identify smaller causal variants more comprehensively.

In particular, carrier screening for recessive disease-associated variants is increasingly moving towards whole exome sequencing (WES) to detect single-nucleotide variants and small indels, forgoing broad CNV analysis. This is concerning for several serious genetic disorders, such as Duchenne muscular dystrophy (DMD), where a large proportion of disease-causing mutations are copy number variants. In DMD (and the milder form, Becker muscular dystrophy) approximately 78% of inherited causal mutations are copy number variants encompassing one or more exons in the DMD gene located on the X-chromosome.

To make WES more applicable for subsequent CNV analysis, several groups have worked on developing computational methods which can use targeted sequencing data to identify copy number variants. However, although there have been some attempts to use these computational techniques in a clinical setting, a variety of limitations prevent most from being directly applicable to carrier screening.

Several of these methods focus on detecting larger CNVs in the context of tumor cell line studies, where factors like normal-cell contamination can affect identification and matched-normal samples are available. Others rely on non-parametric models and are designed for large scale population studies. Only a few have reported sensitivity and specificity levels for individual genes comparable to the levels obtained through microarray and assay methods. In contrast, genetic carrier screening involves genotyping without normal matches and typically provides only a small cohort of reference samples. Most of all, it requires a consistently high degree of sensitivity and specificity for both rare and common CNVs, even when only a small number of specific genes are being screened.

Genetic disorders may be categorized as single-gene (Mendelian) disorders in which the DNA sequence of a gene has errors/mutations; chromosomal disorders in which whole or parts of chromosomes are damaged or missing; or complex disorders involving mutations in two or more genes and environmental factors/lifestyle. A “draft” reference genome sequence for humans, which is a composite sequence, was completed by sequencing and mapping all of the genes, i.e., genome, by the Human Genome Project in 2001.

Sequencing of human genomes enables the identification of genetic variants, including mutations that cause diseases. Exons are protein-coding nucleotide sequences of a gene, i.e., DNA base pair sequences that are transcribed into mRNA and in which the corresponding mRNA molecules are translated into a polypeptide chain specified by the gene. An exome is a sequence of all exons in the genome and comprises about 1% of the human genome or approximately 30 Mb, which is split across approximately 180,000 exons. A protein consists of one or more polypeptide chains that perform a function, such as initiating and performing DNA synthesis, catalyzing metabolic reactions, transporting molecules, and cell signaling.

The emergence of Next-Generation DNA Sequencing (NGS) technology, known as high-throughput sequencing, allows rapid whole genome sequencing (WGS) and “targeted resequencing” of specific areas of interest, such as subsets of genes, including the exome, specific genes of interest, targets within genes and mitochondrial DNA. Whole exome sequencing (WES) consists of selecting only the subset of DNA sequences that encodes proteins and sequencing the exonic DNA using high-throughput sequencing. WES covers more than 95% of the exons. WES uses previous knowledge of the location and sequence of features to target them. In contrast, WGS covers the entire genome.

In 2011, the first successful use of WES to diagnose and alter treatment in an individual child with a life-threatening but previously undefined form of inflammatory bowel disease, was reported. After sequencing, 16,124 variants were identified. Subsequent analysis identified a novel, hemizygous missense mutation, a G to A substitution at a highly conserved position in the X-linked inhibitor of apoptosis gene (XIAP), resulting in substituting a tyrosine for a highly conserved and functionally important cysteine. Since then, exome sequencing has been used to detect a causative variant in several diseases including: Leber congenital amaurosis, Alzheimer disease, maturity-onset diabetes of the young, high myopia, autosomal recessive polycystic kidney disease, amyotrophic lateral sclerosis, immunodeficiency leading to infection with human herpes virus 8 causing Kaposi Sarcoma, acromelic frontonasal dystois, and a number of cancer predisposition mutations.

Although methods for identifying point mutations and small insertions or deletions in genomic DNA are well established, the detection of larger (>100 bp) genomic duplications or deletions of a few kilobases (1000 bases) can be more challenging. Polymerase chain reaction (PCR) is used as a first step in most mutation scanning methods, however, subsequent analyses are generally qualitative rather than quantitative. Without quantitation (molar quantitation) or semi-quantitation (reporting gene dosage relative to an internal standard), heterozygous deletions and duplications may be overlooked, and thus be under-ascertained, e.g., in gene dosage methods, such as fluorescence in situ hybridization (FISH), PCR-based approaches (Multiplex ligation-dependent probe amplification (“MLPA”), QF-PCR, QMPSF and real time PCR), comparative genomic hybridization (CGH) and array-CGH.

MLPA and multiplex amplification and probe hybridization (MAPH) are techniques that allow detection of mid-size deletions of a few kb by simultaneously screening for the loss or duplication of up to 40 target sequences and both rely on sequence-specific probe hybridization to genomic DNA, followed by PCR amplification of the hybridized probe, and semi-quantitative analysis of the resulting PCR products.

The MCOLN1 gene spans 14 kb on chromosome 19 and contains 14 exons encoding a 580 amino acid protein termed mucolipin-1. Mutations in this gene can cause Mucolipidosis type IV (MLIV), a neurodegenerative lysosomal storage disorder that occurs in increased frequency in the Ashkenazi Jewish (AJ) population due to the presence of founder mutations (80% of all patients are AJ). In particular, two alleles in this population, a splice site variant found at 0.8% frequency and a deletion mutation present at 0.2% frequency, combine with other mutations to lead to a total carrier frequency of 1.08%. As a result, these mutations are typically genotyped as part of AJ carrier screening.

### SUMMARY

According to some embodiments, there is provided methods and systems for identifying CNVs and the associated genetic diseases caused thereby for carrier screening.

According to some embodiments, there is provided methods and systems for: identifying copy number variants (CNVs) for a genetic disease, generating a prior distribution model for a normal range of proportional read counts for each of a plurality of exons in one or more genes based on a sample set of training genomes sequenced from DNA of subjects not expressing the genetic disease; the prior distribution model comprising a multi-variate logistic normal model in which the normal range of proportional read counts for each exon is specified by its marginal distribution in a random vector; receiving a plurality of read counts for exon targets sequenced from DNA of a subject undergoing screening for a genetic disease; and determining if the subject has read counts for the plurality of exon targets outside of the normal range of the prior distribution model indicative of a CNV carrier status of the genetic disease, wherein when the read counts are above normal, the CNV is a duplication and wherein when the read counts are below normal, the CNV is a deletion.

In some embodiments, a mean vector and covariance matrix determine normal ranges for the normalized counts of the target exons across multiple dimensions of the model. In some embodiments, the prior distribution model may be a non-conjugate logistic normal prior distribution. In some embodiments, the identified CNVs are in one or more exons.

Also, a need exists for methods for detecting genetic mutations, particularly a deletion that eliminates large (e.g., several thousand) base pairs from a gene encoding an essential protein and which deletion large base pair would otherwise not be detected by resequencing protocols designed to search for a single nucleotide polymorphisms (SNPs) and for small insertions or deletions (INDELs). Detection of deleterious genetic variants, such as a several kb base pair deletion in an exome, is needed to identify a carrier (haplotype) of founder mutations, e.g., in prenatal screening; in cases where conventional diagnostics do not explain a patient's symptoms; in the diagnosis of pediatric patients who may not exhibit a full range of symptoms of a genetic disorder; in cases where there is a family history of a specific genetic disorder; in early diagnoses of disorders that are due to the presence of founder mutations; and to influence current and/or future treatment of patients diagnosed with genetic mutations and provide more precise prognoses in these patients. Because of the size of large deletions, current methods require the entire genome to be sequenced to span and identify the deletion, a relatively slow and memory consuming process. As such, there is a need in the art for efficient detection of large deletions (and/or insertions), for example, of length greater than or equal to 1000 base pairs (1 kb) up to the size of a chromosome's arm (˜125 Mb).

According to some embodiments, there is provided methods and systems for: obtaining short read exome sequences of continuous exomes segments of a genome each having a length of base pairs that is less than or equal to a threshold value (e.g., less than 1000 base pairs and typically 150 base pairs); storing a target sequence of a reference founder genome that has a predefined deletion of a reference sequence having a length of base pairs that is relatively larger than the threshold value (e.g., greater than 1000 base pairs), such that a segment positioned after the deletion is shifted to abut a segment positioned prior to the deletion; and detecting instances of short read exome sequences that straddle both the segment positioned after the deletion and the segment positioned prior to the deletion, wherein both segments falling within the relatively shorter length of the short read exome sequences indicates that the relatively larger length of base pairs has been deleted.

According to some embodiments, there is provided methods and systems for: storing a plurality of short read exome sequences of continuous exomes segments of a reference genome each having a length of base pairs that is less than or equal to the threshold value; aligning a plurality of short read exome sequences of a sample genetic sequence from a subject to a plurality of short read exome sequences of continuous exomes segments of a reference genome; tallying each aligned read pair; classifying each tallied read pair as at least one of: (a) an aligned sequence comprising a segment positioned after a deletion that is shifted to abut a segment positioned prior to the deletion; and (b) an aligned short read pair comprising paired ends, the paired ends comprising a first nucleic acid sequence read from one end of the target sequence of the reference founder genome (prior to the deletion) and a second nucleic acid sequence read from an opposite end of the target sequence of the reference founder genome (after the deletion); wherein a classification of at least (a) or (b) represents a deletion haplotype; displaying the classified read pair to a user; and reporting the sample genetic sequence as a carrier when the read pair is classified as at least (a) or (b).

Embodiments according to the invention are in particular disclosed in the attached claims directed to a method and a computer program product, wherein any feature mentioned in one claim category, e.g. method, can be claimed in another claim category, e.g. computer program product, system, storage medium, as well. The dependencies or references back in the attached claims are chosen for formal reasons only. However any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

### BRIEF DESCRIPTION OF THE DRAWINGS

These and other features, aspects, and advantages of the present invention will become better understood with regard to the following description, and accompanying drawings, where:

FIG. 1 schematically illustrates a generative model for aligned read (“fragment”) coverage during sequencing, in accordance with an embodiment. Fragments map to relevant targets during sequencing according to multinomial distribution with parameter p={p₁ . . . pₖ}. It is noted that v is drawn from multivariate normal with parameters μ, Σ, and cᵢ drawn from multivariate discrete uniform distribution with support [0; C].

FIGS. 2(a)-2(d) are graphs of error in multivariate prior parameter estimation, in accordance with an embodiment. Original parameter (mean and covariance) values were derived from representative estimates for 79 targets across DMD (and an additional baseline target) using a cohort of high coverage samples. Each point represents the average error across 5 simulated sets of subjects at the coverage and cohort size indicated (FIG. 2(a)) shows percent error averaged across μ; (FIG. 2(b)) shows percent error averaged across the expected normalized xᵢ values; (FIG. 2(c)) shows percent error averaged across Σ; (FIG. 2(d)) shows the median percent error across terms in Σ. Legend values indicate total fragment counts (including baseline targets) for each simulated subject.

FIGS. 3(a)-3(d) graphically depict classification performance with increasing fragment coverage, in accordance with an embodiment. Individual subject target intensities for 9 simulated subjects were generated from prior parameters estimated from a cohort of control research subjects. True copy number states from 9 Coriell research subjects (eight with CNVs and one negative control) were used to set multinomial probabilities before fragment coverage simulation. Figures indicate the average number of simulated fragments mapping to the relevant exon targets (not including the baseline targets). FIG. 3(a) and FIG. 3(b) indicate classification performance under the credible interval cutoffs of 0.99 and 0.9 respectively (i.e. targets where the highest-density interval of the chosen size overlaps two copy number states are not assigned a call). Certainty refers to the proportion of targets assigned a final copy number call. FIG. 3(c) and FIG. 3(d) display the copy number state visualization produced after MCMC simulation. FIG. 3(c) indicates a typical result using a low fragment coverage (750 total fragments). The underlying copy number states are unidentifiable. FIG. 3(d) shows results for a sample with the same true copy number states as FIG. 3(c) but a total fragment coverage of 45000 (approximately 20700 at the targets of interest).

FIGS. 4(a)-4(b) are graphs of the sensitivity and specificity trade-offs as cutoff and threshold vary, in accordance with an embodiment. Exon-level classification performance of geneCNV model on nine Coriell research samples, after prior parameter training on 38 research samples was demonstrated. Among the eight samples heterozygous for CNVs, there were a total of 77 affected exons and 634 unaffected exons, used in calculating sensitivity and specificity respectively. FIG. 4(a) shows the effects of varying the credible interval cutoff on the proportion of certain calls, true positives (sensitivity), and true negatives (specificity) for this test set. Exons where the highest-density interval of the chosen cutoff size spans two copy number states are given an “uncertain” call and not included in subsequent sensitivity and specificity analysis. FIG. 4(b) shows the effects of varying the threshold for abnormal copy number state probability (as defined in Methods) on sensitivity and specificity. Note that every exon is given a copy number call using this schema.

FIG. 5 shows a pairwise sample correlation for normalized DMD target coverage, in accordance with an embodiment. Coverage across DMD exons was computed for 60 samples sequenced with two distinct capture sets (one as described in Methods, one with an older version of the TSO panel). Individual target coverage was then normalized by total gene coverage and sample-to-sample correlations were calculated pairwise.

FIGS. 6(a)-6(b) schematically illustrate fragment coverage for training and test samples, in accordance with an embodiment. FIG. 6(a) is a graphical summary of coverage across targets (DMD exons only) for 38 training samples. FIG. 6(b) is a graphical summary of coverage across targets (DMD exons only) for 15 test samples.

FIG. 7 schematically illustrates covariance estimation error, in accordance with an embodiment. Plot of typical percent error across covariance matrix with k=79, estimated from 35 simulated samples at coverage of 60,000 fragments. Original covariance values derived from parameter estimates using training set of 38 samples as described in Methods. Target names represent primary transcript and additional (non-primary) exons in DMD. Note that a small number of covariance terms may have high proportional error; the position of these terms is not consistent between different simulated instantiations of training cohorts.

FIGS. 8(a)-8(c) are graphs of covariance estimation error distributions, in accordance with an embodiment. FIG. 8(a) shows distribution of covariance error proportions (excluding extreme outliers and distribution tail ends). 80% of all covariance terms are contained in this section of the distribution. FIG. 8(b) depicts a plot showing inverse relationship between true covariance values and percent error in estimated values. Lower values are more likely to have higher proportional error.

FIGS. 9(a)-9(b) are graphs of estimation error with target number, in accordance with an embodiment. Plot showing average percent error in Σ (FIG. 9(a)) and μ (FIG. 9(b)) (FIG. 9(a)) as the number of dimensions (targets) increases. At each target number k, mean vector and covariance matrix of the appropriate size (k−1) and (k−1)×(k−1) were generated. One hundred samples with 500 reads/target were simulated using the true parameters, and used to recover the original values. Average error in covariance increases as the number of targets increases, though average error in mean does not correlate with number of targets.

FIGS. 10(a)-(c) show CNV identification in male research subjects, in accordance with an embodiment. Results for male research subjects using geneCNV trained on 38 female subject samples. FIG. 10(a) shows CNV identification in a subject with known deletion in exons 49-52 (designated 28-31 in output). FIG. 10(b) shows CNV identification in a subject with known duplication in exons 2-30 (designated 50-78 in output). FIG. 10(c) shows no CNV identification in a subject with no known CNVs.

FIG. 11 represents a visualization of reads from a heterozygote carrier of a large founder deletion, the MCOLN1 deletion 3′ breakpoint, detected by short read exome sequencing, in accordance with an embodiment.

FIG. 12 represents a similar visualization as FIG. 2 but shows reads mapping to the opposite side of the deletion (MCOLN1 deletion 5′ junction), in accordance with an embodiment.

FIG. 13 schematically illustrates a distribution of sequencing coverage of the 3′ breakpoint across 123 carrier negative samples, in accordance with an embodiment. Only 5 samples had coverage levels below the thresholds of a minimum coverage of 35 read pairs. Sequencing coverage (or “coverage”) may refer to an average number of reads that align to, or “cover,” known reference bases. The next-generation sequencing coverage level often determines whether variant discovery can be made with a certain degree of confidence at particular base positions. Sequencing coverage requirements may vary by application. At higher levels of coverage, each base is covered by a greater number of aligned sequence reads, so base calls can be made with a relatively higher degree of confidence.

FIG. 14 shows an example command and output when run on a known carrier, in accordance with an embodiment.

FIG. 15 schematically illustrates a system for sequencing, aligning, and analyzing one or more genomes to identify copy number variants (CNVs) for a genetic disease, in accordance with an embodiment.

FIG. 16 schematically illustrates a system for identifying copy number variants (CNVs) for a genetic disease, in accordance with an embodiment.

Note that for purposes of clarity, only one of each item corresponding to a reference numeral is included in most Figures, but when implemented multiple instances of any or all of the depicted modules may be employed, as will be appreciated by those of skill in the art.

### DETAILED DESCRIPTION

In the following detailed description, numerous specific details are set forth in order to provide a thorough understanding of the herein provided methods and systems for identifying copy number variants (CNVs) for a genetic disease. However, it will be understood by those skilled in the art that embodiments described herein may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments are not limited in this regard, discussions utilizing terms such as, for example, “processing”, “computing”,” “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently.

Sequencing coverage (or “coverage”) describes the average number of reads that align to, or “cover,” known reference bases. The next-generation sequencing coverage level often determines whether variant discovery can be made with a certain degree of confidence at particular base positions. Sequencing coverage requirements may vary by application. At higher levels of coverage, each base is covered by a greater number of aligned sequence reads, so base calls can be made with a higher degree of confidence.

Some embodiments provide methods and systems for identifying copy number variants (CNVs) for a genetic disease, thereby identifying a carrier status for a mutated gene of interest which is a non-functional gene. In some embodiments, the carrier status is heterozygous for CNVs.

A functional gene may include a gene that fully performs its expected and/or intended function. A non-functional gene may include a gene which, due to gene mutation, such as deletion or duplication, etc., does not fully perform its expected and/or intended function. Any gene which is not fully functional, e.g., a gene which is completely non-functional and/or a gene which is only partially functional with respect to a genetically similar fully functional gene, is referred to herein as non-functional. By way of example, as part of its expected/intended function, the DMD (Dystrophin) gene spans a genomic range of over 2 Mb and provides instructions for making a large protein called dystrophin which contains an N-terminal actin-binding domain and multiple spectrin repeats. This protein is located primarily in muscles used for movement (skeletal muscles) and in heart (cardiac) muscle. Small amounts of dystrophin are present in nerve cells in the brain. While the function of dystrophin in nerve cells remains unknown, research suggests that this protein is important for the normal structure and function of synapses, which are specialized connections between nerve cells where cell-to-cell communication occurs. Dystrophin is a component of a protein complex, the dystrophin-glycoprotein complex (DGC), which bridges the inner cytoskeleton (each muscle cell's structural framework) and the extracellular matrix (the lattice of proteins and other molecules outside the cell), anchoring the extracellular matrix to the cytoskeleton via F-actin. The group of proteins in the DCG work together to strengthen muscle fibers in skeletal and cardiac muscles and protect them from injury as muscles contract and relax. The dystrophin complex may also play a role in cell signaling by interacting with proteins that send and receive chemical signals.

To overcome the aforementioned limitations, embodiments provide a parametric approach for detecting exon-level CNVs in a test sample, which uses a generative model for read depth data across targets in a small number of genes. Embodiments model read depth across these targets as multinomially distributed. This avoids having to explicitly correct for differences in capture efficiency and coverage biases caused by exon length or GC content across targets. To make the model more robust to the inherent variability in library preparation and sequencing, a non-conjugate logistic-normal prior distribution was incorporate into the model. A Markov Chain Monte Carlo (MCMC) approach was implemented in order to estimate posterior distributions for various copy number states across targets in the genes of interest. Like other techniques, the present approach relied on read depth counts in a set of reference samples, specifically for estimation of the prior distribution parameters. These reference samples were assumed not to carry CNVs in the genes of interest and had to be sequenced using the same pipeline as the samples that were tested.

Currently, DMD is not included in typical carrier screening, likely because of the additional processing required for CNV analysis. Embodiments however provide methods and systems for efficiently and accurately identifying CNVs using a parametric model and exome sequencing data. Re-using exome sequencing data reduces memory storage and computational time for detecting CNVs, reducing the overhead associated with CNV analysis.

In an embodiment, provided herein is a method for identifying copy number variants (CNVs) for a genetic disease, the method comprising: generating a prior distribution model for a normal range of proportional read counts for each of a plurality of exons in one or more genes based on a sample set of training genomes sequenced from DNA of subjects not expressing the genetic disease; the prior distribution model comprising a multi-variate logistic normal model in which the normal range of proportional read counts for each exon is specified by its marginal distribution in the random vector; receiving a plurality of read counts for exon targets sequenced from DNA of a subject undergoing screening for a genetic disease; and determining if the subject has read counts for the plurality of exon targets outside of the normal range of the prior distribution model indicative of a CNV carrier status of the genetic disease, wherein when the read counts are above normal, the CNV is a duplication and wherein when the read counts are below normal, the CNV is a deletion. The herein provided methods and systems may be used to identify CNVs at any stage of development, including from pre-conception, in utero, neonatal and in live births of any age.

In some embodiments, a mean vector and covariance matrix determine normal ranges for the normalized counts of the target exons across multiple dimensions of the model. In various embodiments, the method further comprises incorporating a non-conjugate logistic normal prior distribution. In other embodiments of the method, the identified CNVs are in one or more exon.

In some embodiments, the method incorporating a covariance matrix as described above links the normal ranges for normalized counts of independent target exons through the off-target covariance matrix terms. This model more accurately reflects a biological or sequencing-related correlation or interdependence between read counts of a plurality of different target exons, such as that caused by similar GC nucleotide content of different target exons. While this covariance matrix may introduce increased computational load and processing time during the sampling iterations necessary for CNV identification, this load may be modulated or minimized. To reduce this processing time over multiple iterations, a set of conditional covariance matrix components are precomputed and stored in memory before iterations begin, reducing the amount of time necessary for covariance calculations at each iteration.

Methods, systems, and software programs in accordance with some embodiment identify CNVs as the causative mutations of genetic disorders/diseases. In various embodiments, the genetic disorder is Duchenne muscular dystrophy, Becker muscular dystrophy, or any other CNV associated disorder. The method, system and software program identify CNVs for a genetic disease, and thus, detect a carrier status of the CNVs of one or more exons in a gene of interest.

### Example—DMD

### A Generative Model for Read Depth Data

In analyzing the proportion of fragments mapping to each target of interest in DMD, a significant correlation was found between samples processed using the same sequencing pipeline. Based on this, a generative model is developed, which treats target fragment counts as drawn from a multinomial distribution. Then, to explicitly account for both the similarities and sample-to-sample variations across fragment count ratios, a non-conjugate prior distribution for the multinomial probabilities is incorporated. Instead of a conjugate Dirichlet prior, a multivariate logistic-normal distribution has been applied to account for any potential inter-target covariation, in addition to variation within a single target.

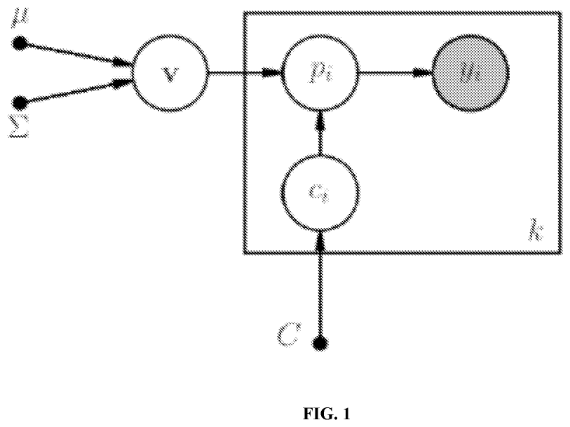

FIG. 1 is a conceptual diagram illustrating a multi-variate logistic normal model graphically. FIG. 1 illustrates latent copy number states and latent target intensities, which together define the overall target mapping probabilities, in accordance with an embodiment. In FIG. 1, k represents the number of targets of interest; yᵢ represents the number of fragments mapping to the i-th target; pᵢ represents the probability of fragment mapping to the i-th target i; cᵢ represents a copy number state of the i-th target; v represents a vector of unnormalized intensities for a plurality of targets; and C represents a vector of cᵢ.

The value of an un-normalized intensity for the i-th target, xᵢ, for each sample may be generated according to a multivariate logistic-normal process, e.g., as follows:

$$1. v = \{ {v}_{1} , … {v}_{k - 1} \} ∼ M V N ( μ , Σ )$$

$$2. {v}_{k} = 0$$

$$3. {x}_{i} = \frac{exp {v}_{i}}{{Σ}_{i = 1}^{k} exp {v}_{i}}$$

Thus, the prior distribution is fully specified by μ and Σ, which have dimension k−1 and k−1×k−1, respectively (e.g., for identifiability the last target intensity is kept constant). Defining the copy number state at each target as cᵢ, the fragment counts Y={y₁, . . . yₖ} for each sample may be represented, for example, as:

$$Y ∼ Mult ( p ) where {p}_{i} = \frac{{c}_{{i}^{x} i}}{\sum {c}_{{i}^{x} i}}$$

For the copy number states, a discrete support representing the possible number of target copies (0,1,2,3) is specified. In some embodiments, to keep the model's sensitivity high, a prior for the copy number states biased towards either 1 (for males) or 2 (for females) may not be introduced, and instead a discrete uniform prior may be used. The unnormalized joint distribution corresponding to this model then becomes, for example:

$$P r ( C , Y , v ; μ , Σ ) ∝ exp ( - 0 .5 {( v - μ )}^{′} {Σ}^{- 1} ( v - μ ) ) {( \frac{1}{\sum {c}_{i} exp {v}_{i}} )}^{R} {\prod }_{i = 1}^{k} {( {c}_{i} exp {v}_{i} )}^{{y}_{i}}$$

where R=Σᵢyi represents the total number of reads in Y.

### Hyperparameter Estimation

An expectation maximization algorithm to fit the mean and covariance of the multivariate logistic-normal distribution based on fragment counts from (e.g., 38) training samples was implemented. This iterative process alternates between maximizing the conditional likelihood Pr(νf|Y,μ,Σ) for each sample (to find the conditional mode of each ν), and then maximizing the expectation of this likelihood with respect to μ and Σ. Thus the first step maximizes, e.g., the following conditional likelihood:

$$\frac{exp ( {Σ}_{i = 1}^{k} {v}_{i} {y}_{i} )}{{( {Σ}_{j = 1}^{k} exp {v}_{j} )}^{R}} exp ( - \frac{1}{2} {( v - {μ}_{a} )}^{T} {\sum }_{a}^{- 1} ( v - {μ}_{a} ) ) \qquad ( 1 )$$

where μₐ and Σₐ are the values generated by the previous expectation maximization (EM) step. Then subsequent values (μₐ₊₁,Σₐ₊₁) are estimated, e.g., as:

$${argmax}_{μ , Σ} {\sum }_{i = 1}^{m} 𝔼 [ log Pr ( {v}_{i} \vert μ , Σ ) \vert {Y}_{i} , {μ}_{a} , {Σ}_{a} ]$$

where m is the number of training samples. This is approximated, e.g., by minimizing:

m log|E|+Σᵢ₌₁^{m}(\hat{μ}ᵢ−μ)^{T}Σ⁻¹(\hat{μ}ᵢ−μ)+Σᵢ₌₁^{m}trace(Σ⁻¹\hat{Σ}ₗ) (2)

This simplification takes advantage of the expectation of a quadratic form and the following multivariate normal approximation to the conditional likelihood, for example:

Pr(ν|Y,μ,Σ)≈MVN(\hat{μ},\hat{Σ})

where \hat{μ} is the conditional mode of ν and \hat{Σ} is the negative inverse Hessian at the mode. Finally, equation (2) is minimized for example by:

$${μ}_{a + 1} = \frac{1}{m} {\sum }_{i = 1}^{m} {\hat{μ}}_{i} and$$

$${Σ}_{a + 1} = \frac{1}{m} {\sum }_{i = 1}^{m} [ ( {\hat{μ}}_{i} - {μ}_{a + 1} ) {( {\hat{μ}}_{i} - {μ}_{a + 1} )}^{T} + {\hat{Σ}}_{i}$$

### Inferring Copy Number States

### MCMC

Given the unnormalized joint distribution above and estimated hyperparameters, the true joint distribution can be estimated using a Markov Chain Monte Carlo sampling technique. This then also allows for approximating the marginal posterior probability distributions for the copy number states. Examining the discrete copy number posterior probability distributions provides an intuitive measure of confidence (analogous to a high-density credible interval) that can be used as a decision criterion to make copy number variant calls.

For example, a variation of the Metropolis-within-Gibbs algorithm may be implemented, where at each iteration, and for each target, a new copy number state cᵢ is drawn uniformly from its support and a new target intensity νᵢ conditioned on the most recent values is used for all other targets. To analyze convergence of the algorithm, the Gelman-Rubin potential scale reduction factor (PSRF) may be calculated and tracked for the complete-data log likelihood and the νᵢ values, over steps of (e.g., 5000) iterations and using a coarse optimization over burn-in proportion. As convergence criteria, the standard PSRF threshold of (e.g., 1.1) for the log-likelihood was used and require e.g. at least 80% of νᵢPSRFs to be less than the standard PSRF threshold. After convergence, posterior probability distributions may be calculated over the copy number states for each target from the iteration values.

### Metastability Error Analysis

In addition to Gelman-Rubin convergence analysis, some potential metastability error is accounted for with an additional likelihood comparison step. Metastability error, when an MCMC simulation appears to have converged but has only reached a lower-likelihood metastable state, is caused by multimodality in the joint distribution space. In general, the chance of metastability error may be reduced by running multiple chains and selecting overdispersed initial variable values (inherent in the first convergence analysis step). To further reduce the possibility of metastability error causing false positives, the complete-data log-likelihood (Lₘ) of the combination of most likely copy number states (comprised of the most likely copy number state in the posterior at each target) may be compared with the complete-data log-likelihood (Lₙ) of the “normal” copy number state. For instance, in females, this would mean e.g. cᵢ=2 for all targets. (Before comparison, the log-likelihoods may be optimized with respect to target intensities, holding the copy number states constant at the values described above.) If Lₙ is significantly larger than Lₘ, indicating metastability error, the MCMC simulation may be repeated, until the difference Lₘ−Lₙ surpasses a minimum (e.g., user-defined) threshold.

### Absolute Copy Number Identification

Since the provided generative model typically cannot identify the absolute copy number state when all targets have equal copy number (as the relative frequency of all targets is equivalent), “baseline” targets may be incorporated, which are assumed to be consistently representative of the normal genome-wide copy number. In an example study using a sequencing pipeline, 20 such genes were identified based on criteria including consistent average coverage across samples. For this study, seven of these genes were selected for a total of 112 additional “baseline” targets, which were included in the model and fragment counts as a single aggregated baseline. By including this aggregate baseline along with the targets of interest (thus increasing the dimensions of our hyperparameters and multinomial probability by one), the absolute copy number states of the remaining targets was accurately identified. During MCMC simulation, the copy number state of this aggregate baseline was kept constant and never updated.

### Aggregation and Final Variant Calling

Setting the posterior probability threshold for calling a copy number state not equal to the normal state may help determine the sensitivity and specificity of the test. For the present study, a conservative threshold of e.g. 0.5 was set in order to maximize sensitivity, with a trade-off in specificity. This is equivalent to calling the copy number state with highest probability when the posterior distribution spans two states. Unlike other techniques, no attempt was made to aggregate targets before calling copy number state (through a hidden Markov model or other method), instead calling copy number state for each target individually and afterwards aggregating only those that matched in copy number. This choice was also motivated by the desire to increase sensitivity for small (single- or double-exon) CNVs.

### Sample Collection and Sequencing

In an example embodiment, a total of 42 saliva samples were processed and analyzed, in addition to 11 DNA samples obtained from the Coriell Institute (Coriell Institute for Medical Research, Camden, N.J.). Saliva samples were collected and sequenced on the Illumina platform. The sequencing of the volunteer and Coriell research samples sequenced was performed on a NextSeq 500 sequencing system instead of a MiSeq, and in order to increase the genomic coverage of the DMD gene, samples were enriched with a custom mix-in panel containing a 2:1 ratio of baits from the Illumina TruSight One (TSO) panel (4,813 genes) mixed with the Illumina Inherited Disease Panel capture bait set (a subset of 552 genes).

### Read/Fragment Coverage

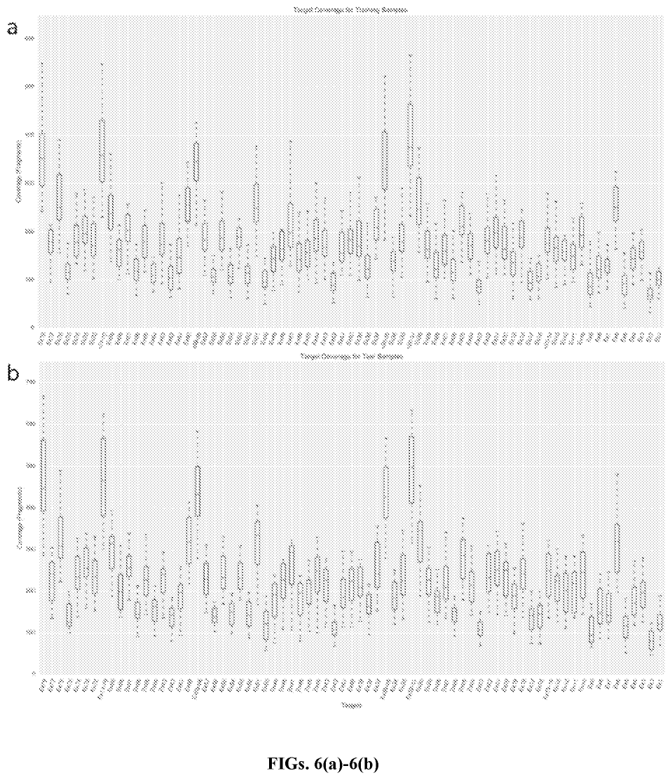

Exon target coordinates were determined based on the intersection of TSO panel bait intervals and exon locations designated by Ensembl database transcripts for hg19 (for DMD transcript ENST00000357033.8, RefSeq NM_004006 was used). Coverage across exon targets was calculated to extract fragment counts from individual BAMs, where each fragment corresponded to a properly mapped pair of reads. Included reads were correctly oriented, with mapping quality e.g. ≥60 and insert length less than a designated merge distance (e.g., 629 bp for DMD). Before computation, exons closer than the designated distance were merged to avoid repeated counting of read pairs that overlapped more than one exon (for proper mapping to individual targets). Reads flagged as PCR duplicates were excluded. In addition, due to insufficient and inconsistent coverage, exon 78 in DMD (chrX: 31144758-31144790) was excluded from all subsequent analysis. Summary coverage across the primary exons of DMD for training and test samples is visualized in FIGS. 6(a)-6(b).

FIGS. 6(a)-6(b) illustrate fragment coverage for training and test samples. FIG. 6(a) is a graphical summary of coverage across targets (DMD exons only) for 38 training samples. FIG. 6(b) is a graphical summary of coverage across targets (DMD exons only) for 15 test samples.

### Selection of Training Samples

In order to train the model and estimate hyperparameter values, geneCNV requires a set of presumed normal samples sequenced using the same pipeline and capture technology. For the validation experiments, 38 volunteer samples were identified that showed similar target coverage (and were sequenced with the same bait set) in training the model. Pairwise sample correlations were examined for normalized coverage across DMD targets in these training samples, in addition to the eight CNV positive validation samples, and 13 samples sequenced with a different bait set.

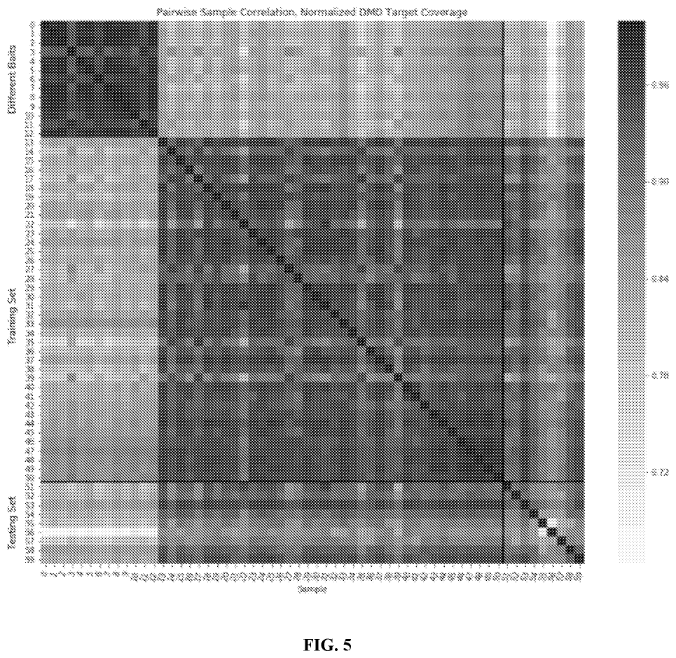

FIG. 5 displays these correlations, demonstrating a relatively high degree of correlation 1 among the training and testing samples, compared to the samples sequenced with a separate bait set. As expected, there is some observable variation even among samples using the same bait set, likely due to different batch-level effects. To estimate model parameters as generally as possible, none of these samples were excluded, though outliers with any pairwise correlations<0.8 were excluded from a training set. In addition, it should be noted that test samples with larger CNVs (such as sample 56, which contains a 29 exon duplication) will show lower levels of overall correlation with other samples.

### MLPA

Copy number states across DMD targets were confirmed for all samples analyzed in the software comparison through multiplex ligation-dependent probe amplification (MLPA). All amplification and processing steps were performed according to MLPA General Protocol and manufacturer protocol for the SALSA MLPA P034 DMD probe mix kit (MRC-Holland, Netherlands). Fragment separation and analysis was performed on the PCR products via capillary electrophoresis on the ABI 3130xl (Applied Biosystems, Foster City, USA). Data files were analyzed with Coffalyser.NET software maintained by MRC-Holland.

### Results

### Theoretical Parameter Estimation Error and Classification Performance

There are several potential sources of error in the present model's ability to accurately call CNVs, including poor estimation of the prior distribution's hyperparameters, and subsequent inference error (of the copy number state probability distributions) introduced during MCMC sampling. As a proof of concept, the expected effects of varying fragment coverage and the number of training samples on the resulting error were quantified using simulated data.

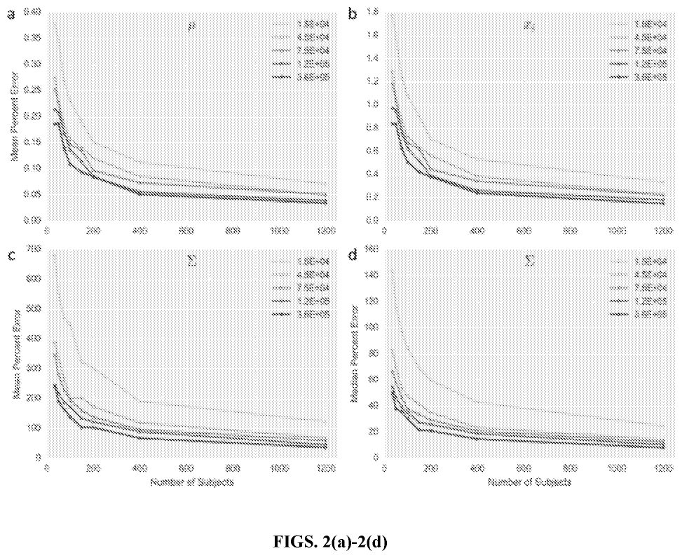

In FIGS. 2(a)-2(d), it is demonstrated how the parameter estimation error decreases as the both the number of samples and the total coverage per sample increases. A single set of representative parameters, derived from mean and covariance values estimated from a cohort of high coverage samples, were considered. Then these parameters were estimated using the EM training algorithm after simulating increasing numbers of fragments for different numbers of samples. Error in estimation of the covariance terms decreased more significantly and consistently compared to error in the mean, though increasing fragment coverage beyond 75,000 fragments led to only a marginal continued decrease in error for both parameters. Similarly, increasing the size of the sample training set beyond 400 samples led to more modest decreases in estimation error of both the mean and covariance terms.

In terms of estimating the logistic-normal mean (and the resulting mean exon intensity values), even using just 35 training samples (and fragment coverage of 45000) could reduce the average percent error in the normalized xᵢ intensities to 1%. However, the percent error in the covariance terms is proportionally much higher, possibly because true covariation between targets (represented in the off-diagonal terms of the matrix) is likely very low on an absolute level. Analyzing the distribution of expected error in the covariance matrix reveals that there is a small number of terms with extremely high proportional error, and in fact, the median percent error is less than 60% for most cohort and coverage levels tested (FIGS. 2(a)-2(d) and FIGS. 7, 8(a)-8(b). Thus, while limiting the mean percent error in the covariance terms to less than 100% would require an unrealistic cohort size and level of coverage for this number of targets, the majority of covariance terms could be estimated to within 80% of their true values with 35 training samples (and fragment coverage of 45000).

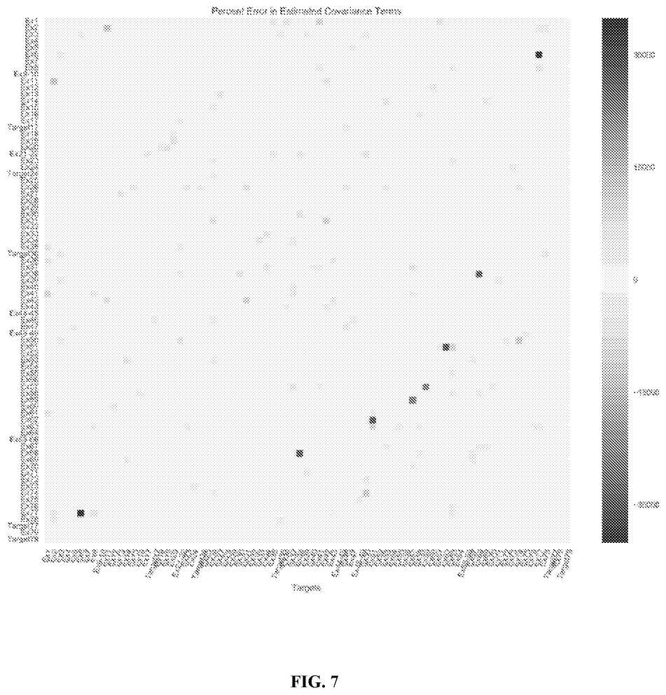

FIG. 7 shows covariance estimation error. Plot of typical percent error across covariance matrix with k=79, estimated from 35 simulated samples at coverage of 60,000 fragments. Original covariance values derived from parameter estimates using training set of 38 samples as described in Methods. Target names represent primary transcript and additional (non-primary) exons in DMD. A small number of covariance terms have very high proportional error; the position of these terms is not consistent between different simulated instantiations of training cohorts.

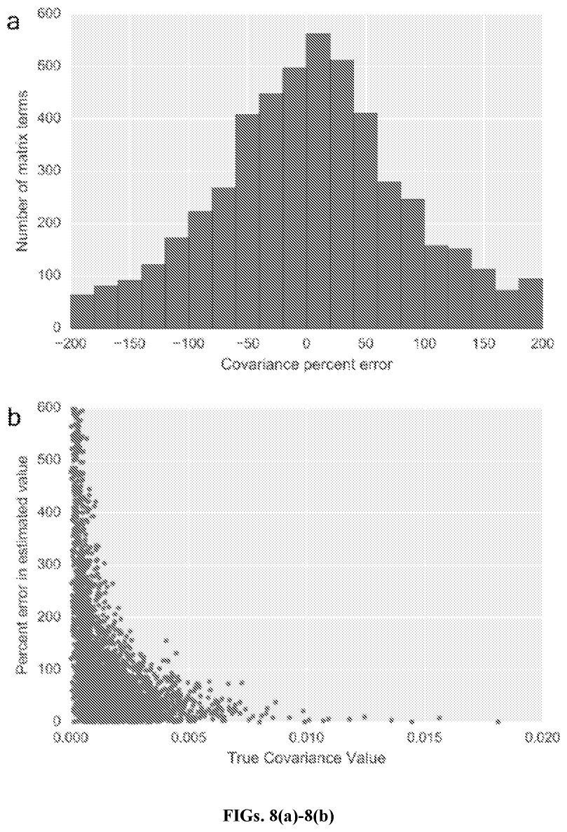

FIGS. 8(a)-8(c) graphically illustrate covariance estimation error distributions. FIG. 8(a) shows distribution of covariance error proportions (excluding extreme outliers and distribution tail ends). 80% of all covariance terms are contained in this section of the distribution. FIG. 8 (b) depicts a plot showing inverse relationship between true covariance values and percent error in estimated values. Lower values are more likely to have higher proportional error.

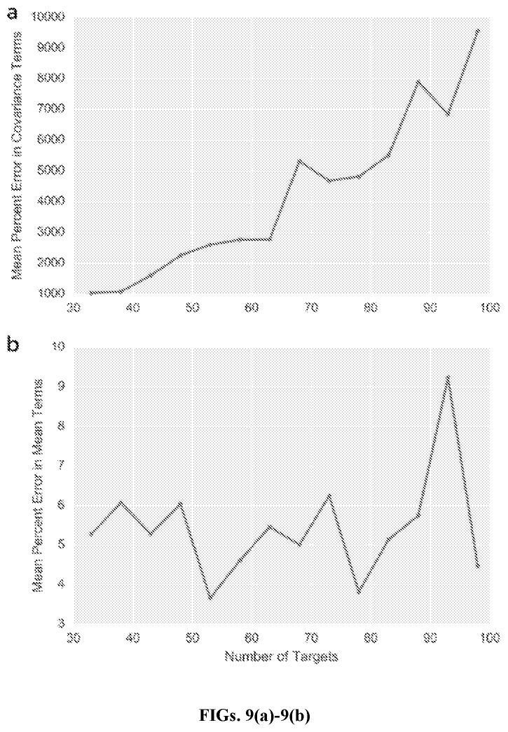

FIGS. 9(a)-9(b) graphically illustrate estimation error with target number, showing average percent error in Σ (FIG. 9(a)) and μ (FIG. 9(b)) (FIG. 9(a)) as the number of dimensions (targets) increases. For each target number k, mean vector and covariance matrix of the appropriate size (k−1) and (k−1)×(k−1) were generated. One hundred samples with 500 reads/target were simulated using the true parameters, and used to recover the original values. Average error in covariance increases as the number of targets increases, though average error in mean does not correlate with number of targets.

Because the original parameters included a term representing the aggregate baseline, the total fragment count includes coverage outside of the main targets of interest (in this scenario, only about 46% of the total fragments map to targets corresponding to exons in the gene of interest). Thus, coverage of 45000 fragments represents coverage at the level of approximately 21000 for a gene similar to DMD. In terms of per-base coverage, this corresponds to an average read depth of about 250. Overall, the analysis indicates that at least 35 training samples with high coverage (>200) across the gene of interest are needed to limit the parameter estimation error (particularly in the covariance terms) to a reasonable amount.

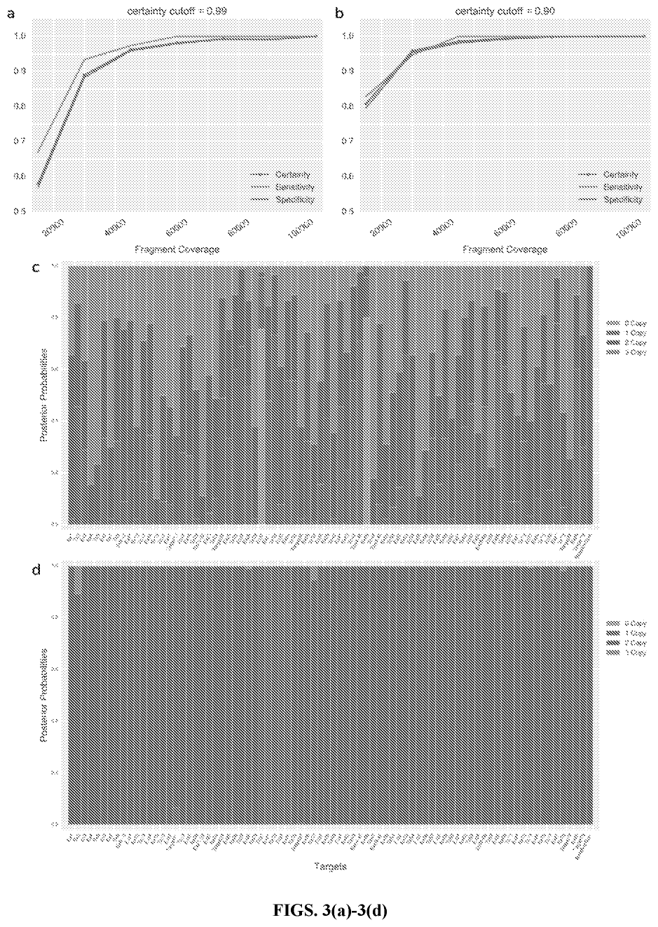

Also investigated was the effect of increasing test sample coverage on the model's ability to identify relative copy number states (FIGS. 3(a)-3(d)). For this experiment, no estimation error in the prior parameters was assumed and all test sample target intensities were generated from the same logistic-normal hyperparameters. Nine different samples were simulated (eight with CNVs corresponding to those found in the Coriell research subjects, and one negative control) with levels of total fragment coverage varying from 15,000 to 105,000. In generating the copy number calls, credible interval cutoffs were used (instead of a threshold as described in Methods supra) to measure the proportion of targets we could call with certainty at each coverage level. This analysis shows that even with a high cutoff, increasing test sample coverage to approximately 45,000 (21,000 for gene of interest) is sufficient to raise exon-level certainty, sensitivity and specificity above 95%, with marginal improvements as coverage increases beyond this level. At a slightly lower cutoff, all three metrics reach 100% at a coverage of 75,000 (34,000 for gene of interest). Thus, assuming the model has very low parameter estimation error, fragment coverage of 21,000 should generate accurate copy number calls.

In addition, FIGS. 3(a)-3(d) demonstrate the behavior of the MCMC simulation results at very different coverage levels. At an extremely low coverage level (750 total fragments), the resulting estimates for the copy number state distributions show a large amount of uncertainty, and the underlying true copy number states are unidentifiable. At a high level of coverage (45,000 fragments total, with 20,700 mapping to the gene of interest), the copy number state distributions clearly indicate the underlying heterozygous deletion of five exons in this sample.

Validation with Samples Heterozygous for CNVs in DMD

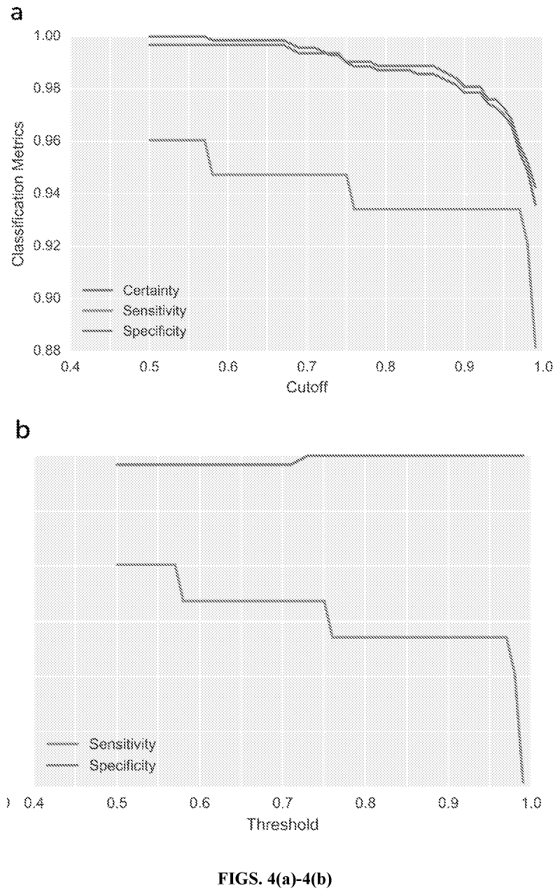

To assess the model's ability to accurately call CNVs in DMD, samples from nine Coriell research subjects were used (eight of which are heterozygous for CNVs of various sizes, ranging from a single exon deletion to a 29 exon duplication). Model hyperparameters were estimated from a set of 38 research subjects sequenced using the same pipeline as the Coriell test subjects (FIGS. 4(a)-4(b)) and Table 4.

FIGS. 4(a)-4(b) illustrate the model's performance at different credible interval cutoff and threshold values. The proportion of certain calls at cutoffs of 0.9 and 0.99 were consistent with our simulation results, given the average DMD fragment coverage (16400) of these nine samples (36000 across DMD and baseline targets). The observed sensitivity and specificity at these cutoff values were also roughly consistent with the simulation results in FIG. 3(a)-3(d), indicating fairly low parameter estimation error from model training. As in the simulation, decreasing the cutoff consistently increased both sensitivity and specificity, though neither sensitivity nor specificity reached 1.0, even at the lowest possible cutoff. This indicated some noise in the final MCMC results (and potentially some error in the hyperparameter estimation), likely due to the lower coverage of these samples.

In calling complete copy number states, a conservative threshold of 0.5 was used instead of a cutoff (to generate calls across all targets), which achieved an exon-level sensitivity of 0.961 and a specificity of 0.997. Of the 77 exons included in the CNVs, 74 were correctly called by our model; the three false negatives were three non-contiguous exons in a 29-exon duplication. At the subject level, where one only has to detect a change in any exons copy number to qualify the subject as a carrier, perfect concordance was observed between the geneCNV analysis and the known carrier statuses for these test samples.

In accordance with an embodiment, a novel computational method is provided for identifying copy number variants from targeted exome sequencing data using a generative Bayesian model. Unlike most other methods, the herein provided generative model is intended to be representative of the underlying reactions, including paired-end read alignment, during a typical hybrid-capture sequencing pipeline. In addition, the method's basis in modeling read alignment on an exon-level allows detection of even small copy number variants (one to two exons in length) with high sensitivity.

Since the present technique models target alignment with a multinomial distribution, an important consideration was the prior distribution for the multinomial parameters. The simulation results indicate that using a multivariate logistic-normal distribution yields accurate copy number identification, especially when the prior parameters are well-estimated and coverage is sufficiently high (e.g., approximately 21,000 fragments across targets of interest, or an average of 275 fragments per exon). The accuracy of the prior parameter estimation is sensitive to the number of samples in the reference set, in addition to these samples' coverage levels. Assuming a similarly high level of coverage, the prior mean can be accurately estimated with only a few e.g. 30 reference samples. The prior covariance can be reasonably estimated with e.g. 30-50 samples, although additional reference samples (and increased coverage) will typically improve parameter estimation.

The utility of the some embodiments was demonstrated as part of a downstream clinical analysis of copy number variation in the context of carrier screening for the DMD gene. GeneCNV was used to detect CNVs in nine Coriell research samples with known carrier statuses (including eight with large deletions or duplications and one negative control). On a subject level, complete concordance was found between the known statuses of these samples (which were also confirmed by MLPA), and the mutation calls generated by the present program. Across the total number of affected and unaffected exons in these nine samples, an overall sensitivity of 0.96 and a specificity of 0.998 was observed, indicating almost complete agreement between geneCNV's mutation calls and actual copy number state on an exon level.

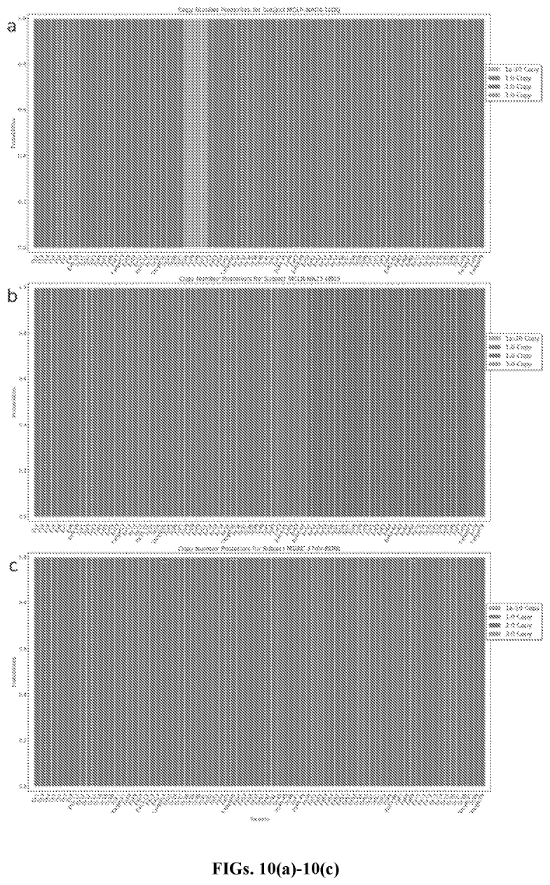

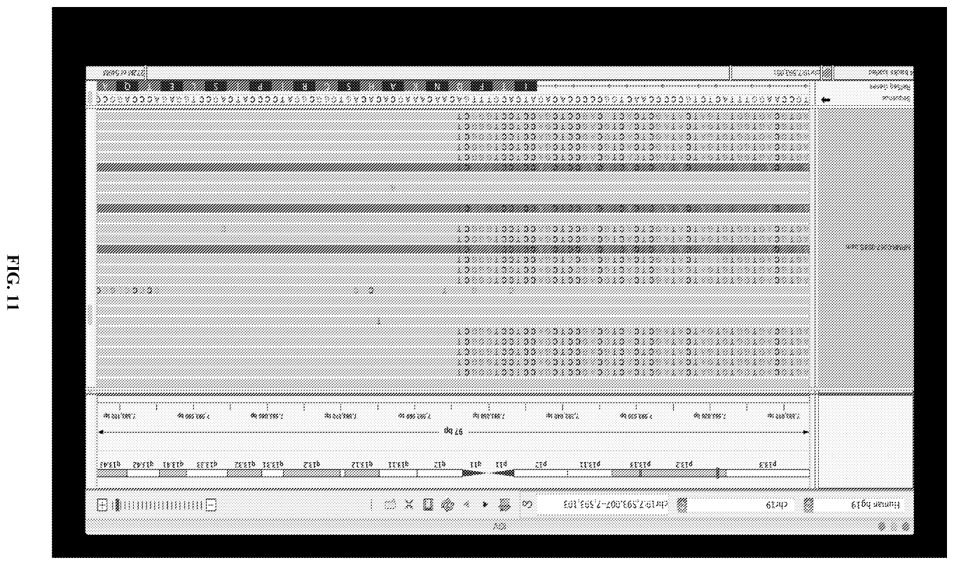

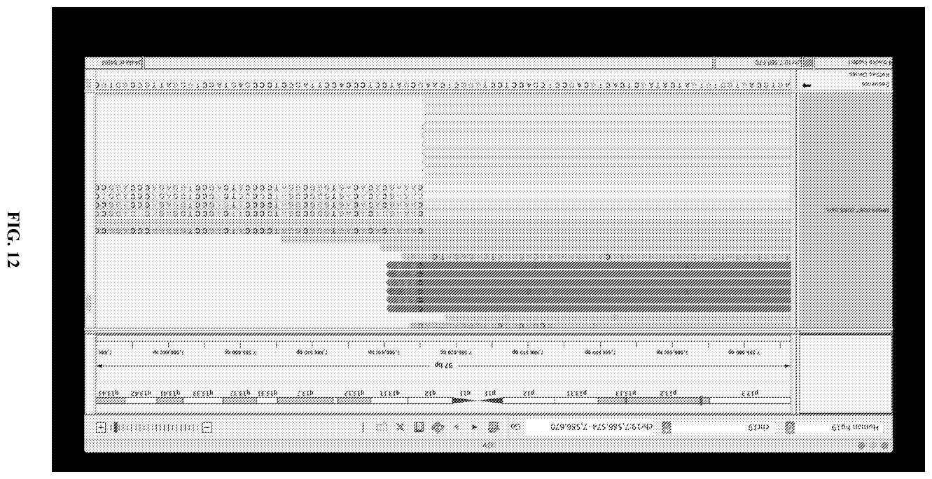

Using geneCNV for clinical CNV analysis in DMD demonstrates another advantage of the model, which allows for testing of targets on the sex chromosomes in addition to autosomal targets. As long as baseline normalization is included, and the model is trained on female samples, absolute copy numbers can be estimated for targets across all chromosomes for both male and female test samples (FIGS. 10(a)-10(c)).

FIGS. 10(a)-10(c) demonstrate CNV identification in male research subjects, showing results for male research subjects using geneCNV trained on 38 female subject samples. FIG. 10(a) shows CNV identification in a subject with known deletion in exons 49-52 (designated 28-31 in output). FIG. 10(b) shows CNV identification in a subject with known duplication in exons 2-30 (designated 50-78 in output). FIG. 10(c) shows no CNV identification in a subject with no known CNVs.

The validation of the computational technique for CNV detection helps expand the potential utility of whole exome and targeted panel sequencing used in carrier screening. This is particularly true for genes like DMD which have thus far been inadequately covered by most existing carrier screens. By incorporating the technique into an existing high-throughput sequencing pipeline, clinicians can more easily conduct accurate CNV analysis for multiple disease-causing genes without relying on additional laboratory assays.

### Deletion Detection

In one aspect, embodiments provide methods and systems for detecting relatively large predefined deletions, known from a previously examined genome, using short read exome sequencing, to identify a carrier status for a gene of interest. Large deletions, by virtue of their lengths that span a continuous sequence of typically thousands of base pairs, are conventionally detected by full-genome sequencing, a time-consuming and cumbersome task. According to embodiments, there is provided a fast and efficient way to detect large deletions using short exome sequencing, which is significantly faster and more memory efficient than full-genome sequencing. Short exome sequencing has conventionally been limited to detecting short deletions (smaller than the short exon length) because the short exons were unable to span the length of relatively longer deletions. However, according to embodiments, short exome sequencing is used to detect large deletions (of greater length than the exon sequences) by detecting short transition regions where the pre-deletion segment and post-deletion segment of the exome join. Although the short exon sequence cannot span the entire length of the deletion, it is able to detect the short transition segment that is the signature of the large deletion. By using short exome sequencing, embodiments provide a concise and fast mechanism to detect large deletions, as compared to conventional full-genome sequencing.

Example large deletions include, but are not limited to, a deletion haplotype of MCOLN1 and a deletion haplotype of CFTR.

Although the AJ splice site mutation of MCOLN1 is a simple SNP found by standard NGS exome sequencing protocols, the deletion mutation removes about 6,450 base pairs from the gene, which is a relatively large predefined deletion in a reference founder genome. The nearly 6.5 kb deletion will not be detected by resequencing protocols designed to look for SNPs and small INDELs. Consequently, alternate PCR assays have been developed to detect them. However, because this deletion spans from 928 bp upstream of exon 1 to the 31st bp of exon 7, reads spanning the deletion that sequence both the 5′ and 3′ breakpoint positions are enriched and sequenced in Exome panels (FIG. 11 (3′ end), FIG. 12 (5′end)) and can be detected by the herein provided methods that looks for this specific deletion. According to some embodiments, a stand-alone software program is provided that, given exome resequencing data, detects such large deletions based on the presence of reads spanning the deletion junction, which have unique signature sequences and inferred insert lengths that can be used to determine if the variant is present.

Embodiments search for read pairs that either sequence across the deletions breakpoints or have component reads which align on opposite sides of the breakpoints (the post-deletion segment which is shifted roughly 6.5 kb compared to a non-carrier reference sequence for the deletion mutant of the MCOLN1 gene). If any such reads are detected, embodiments may identify the associated sample or subject as a carrier. If not, embodiments may verify that sufficient sequencing data is present where the deletion haplotype could have been detected and may classify the subject or sample as carrier negative. Embodiments overcome the limitations of protocols designed to identify a point mutation (e.g., a random SNP), and small INDELs in genomic DNA.

An embodiment may include detecting a relatively large predefined deletion in a reference founder genome using short read exome sequencing by: obtaining short read exome sequences of continuous exomes segments of a genome each having a length of base pairs that is less than or equal to a threshold value; storing a target sequence of a reference founder genome that has a predefined deletion of a reference sequence having a length of base pairs that is relatively larger than the threshold value, such that a segment positioned after the deletion is shifted to abut a segment positioned prior to the deletion; detecting instances of short read exome sequences that straddle both the segment positioned after the deletion and the segment positioned prior to the deletion, wherein both segments falling within the relatively shorter length of the short read exome sequences indicates that the relatively larger length of base pairs has been deleted. The target sequence of the reference founder genome may be referred to as a reference sequence. The reference sequence may include the sequence of the deletion before the deletion occurs, the segment positioned prior to the deletion, and the segment positioned after the deletion.

In an embodiment, the obtained short read exome sequences are a plurality of short read pairs of exome sequencing data from a DNA sample of a subject, the short read pairs comprising paired ends, the paired ends comprising a first nucleic acid sequence read from one end of the target sequence of the reference founder genome and a second nucleic acid sequence read from an opposite end of the target sequence of the reference founder genome. In some embodiments, each of the first nucleic acid sequence read and the second nucleic acid sequence read is on an opposite side of a deletion junction of the deletion, in a known positional relationship in the reference founder genome. The reference founder genome may comprise a wild type nucleic acid sequence without any predefined deletions.

In some embodiments, each of the first nucleic acid sequence read and the second nucleic acid sequence read comprises less than 1000 nucleic acid base pairs, and for example, approximately 150 nucleic acid base pairs.

In some embodiments, the target sequence of the reference founder genome comprises a nucleic acid sequence created by a base pair deletion on either side of a deletion junction in an exome of the gene of interest. In some embodiments, the nucleic acid sequence spans a 3′ breakpoint position in the gene of interest.

In some embodiments, nucleic acid sequences of the plurality of short read pairs of exome sequencing data may be aligned with the stored target sequence of the reference founder genome to obtain a matched alignment of short read pairs of exome sequencing data to the stored target sequence of the reference founder genome. In some embodiments, a visualization may be provided of the matched alignment of short read pairs of exome sequencing data to the stored target sequence of the reference founder genome.

In an embodiment, the matched alignment of the short read pairs of exome sequencing data comprises an aligned first nucleic acid sequence read and an aligned second nucleic acid sequence read, each nucleic acid sequence read begins on either side of the deletion junction and each of the first and second nucleic acid sequence read does not comprise a deletion junction sequence. In some embodiments, the aligned first nucleic acid sequence read and the aligned second nucleic acid sequence read may be aligned with an expected nucleic acid deletion sequence for the gene of interest. A matched realignment to the expected nucleic acid deletion sequence may confirm the subject is a heterozygous carrier of the large base pair deletion. In some embodiments, short read pairs are mapped to within 2 kb of the deletion junction. In further embodiments, short read pairs are mapped to within 500 base pairs of the deletion junction.

In an embodiment, the relatively large predefined deletions of the reference founder genome comprise from a 125,000,000 base pair deletion to a 1,000 base pair deletion. In another embodiment, the relatively large predefined deletions of the reference founder genome comprise a 6,500 base pair deletion. In some embodiments, the 6,500 base pair is deleted from the MCOLN1 gene. In some embodiments, an absence of a matched alignment of short read pairs of exome sequencing data comprising at least 8 base pairs on either side of the deletion junction is required in a minimum of 35 short read pairs to determine deletion is not present in the DNA sample.

A functional gene may refer to a gene that fully performs its expected and/or intended function. A non-functional gene may refer to a gene which, due to gene mutation, such as deletion or duplication, does not fully perform its expected and/or intended function. Any gene which is not fully functional, e.g., a gene which is completely non-functional and/or a gene which is only partially functional with respect to a genetically similar fully functional gene, may be referred to herein as non-functional. By way of example, as part of its expected/intended function, the Mucolipin 1 gene (MCOLN1) provides instructions for making a protein called mucolipin-1. This gene encodes a member of the transient receptor potential (TRP) cation channel gene family.

Mucolipin-1 is located in the membranes of lysosomes and endosomes, compartments within the cell that digest and recycle materials. Mucolipin-1 plays a role in the transport (trafficking) of fats (lipids) and proteins between lysosomes and endosomes. This protein acts as a channel, allowing positively charged atoms (cations) to cross the membranes of lysosomes and endosomes. The channel is permeable to Ca(2+), Fe(2+), Na(+), K(+), and H(+), and is modulated by changes in Ca(2+) concentration. Mucolipin-1 is important for the development and maintenance of the brain and light-sensitive tissue at the back of the eye (retina). In addition, this protein is likely critical for normal functioning of the cells in the stomach that produce digestive acids. Mucolipin-1 is ubiquitously expressed in spleen (RPKM 28.6), adrenal (RPKM 14.9) and 24 other tissues.

By way of another example, the cystic fibrosis transmembrane conductance regulator gene (CFTR), as part of its expected/intended function, provides instructions for making, a protein called the cystic fibrosis transmembrane conductance regulator. The CFTR protein functions as a channel across the membrane of cells that produce mucus, sweat, saliva, tears, and digestive enzymes; the channel transports negatively charged particles called chloride ions into and out of cells. Transport of chloride ions helps control the movement of water in tissues, which is required for the production of thin, freely flowing mucus, which is a slippery substance that lubricates and protects the lining of the airways, digestive system, reproductive system, and other organs and tissues. The CFTR protein also regulates the function of other channels, such as those that transport positively charged particles called sodium ions across cell membranes; these channels are required for the normal function of organs such as the lungs and pancreas.

FIG. 11 is a visualization of the short exome reads from a heterozygote carrier of the MCOLN1 deletion 3′ breakpoint. The visualization is generated by an Integrated Genome Viewer (IGV). Reads spanning the junction of the deletion align to exon 7, predefined in a founder sequence, and targeted for analysis for detection of the mutation. Reads matching a reference genome (at bottom) are omitted; nucleotides that differ from the reference genome bases are specified.

FIG. 12 is a similar visualization as FIG. 11 of the short exome reads from a heterozygote carrier of the MCOLN1 deletion, but shows reads mapping to the opposite side of the deletion (MCOLN1 deletion 5′ junction).

In some embodiments, reads having paired ends that begin on opposite sides of a deletion, as shown in FIGS. 11 and 12, even if the junction sequence is not present in the reads, represent the deletion haplotype. A classified read pair in such a sample may be reported as a carrier for the deletion in MCOLN1, known to cause the recessive genetic disease Mucolipidosis type IV. In some embodiments, the visualizations of reads on opposite sides of the deletion is performed on a computer (e.g., system server 110) having one or more processors (e.g., server processor 115), one or more memories (e.g., server memory 125), and one or more code sets or software (e.g., server module(s) 130) stored in the memory and executed by the processor.

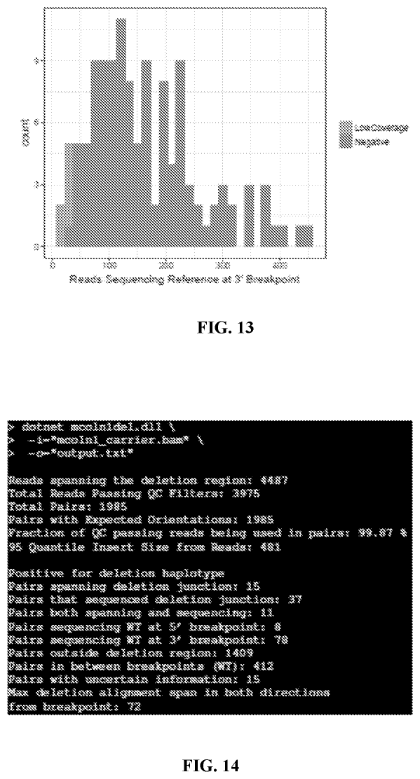

FIG. 13 is a graph of a distribution of sequencing coverage of the 3′ breakpoint across 123 carrier negative samples. Only 5 samples had coverage levels below the thresholds of a minimum coverage of 35 read pairs.

FIG. 14 shows an example command and output for a known carrier of a MCOLN1 deletion.

Some embodiments overcome the aforementioned limitations of protocols/methods by identifying large deletions using short exome sequencing previously designed only to identify a point mutation (i.e., a random SNP), and small INDELs in genomic DNA. Therefore, embodiments reduce unnecessary processing power and memory usage by enabling a deletion haplotype (e.g., of a gene of interest, such as, MCOLN1) carrier status to be determined by using data from NGS screens, without requiring the extensive processing power and memory usage associated with full-genome sequencing.

### Example—MCOLN1

### Method Assumptions

Some embodiments may assume that the genomic region spanning the deletion has been enriched for using a capture panel containing the MCOLN1 gene (such as the Illumina TruSight One or Inherited Disease panels), and that the (e.g., FASTQ) read data is aligned using the program bwa mem (http://bio-bwa.sourceforge.net/bwa.shtml). For reference, on hg19, the 1-based coordinates of this deletion (when left aligned as there are three bases, CAA, that can be ambiguously placed), removes the bases [7586622,7593055]. This deletion is referred to by multiple names, including: ‘511del643’, ‘g.7586625_7593057del’ and ‘c.1_788del’. In some embodiments, the input BAM file contains data from only one individual.

### Collecting Read Pairs Spanning MCOLN1

Given dataset stored in a (e.g., BAM) file containing paired-end sequencing data, the program first verifies that Chromosome 19 is the expected size for the HG19 reference and then parses out all the reads that match the following conditions:

(1) The read is mapped to within a predefined distance (e.g., 500) of basepairs of the region spanned by the deletion, e.g., [7586622-500, 7593055+500].

(2) SAM flags for the read may match the following conditions:
(a) Not a duplicate (0×0400)
(b) Not a QC failure (0×0200)
(c) Paired read (0×0001)
(d) Not secondary alignment (0×0100)

Reads that pass these conditions may then be joined by matching read names into read pairs for analysis. If a read is not paired with a match or if the two reads in a pair do not map to opposite strands on the reference sequence, the data may be ignored or discarded.

To verify the data, when parsing the reads, some embodiments verify that the typical insert size of read pairs passing the above conditions is not too large (e.g., 95th quantile<1000 bp) and/or that the number of the original reads that passed filters and were converted into read pairs is not less than a predetermined threshold (e.g., 80%) of all the reads spanning the coordinates queried in the dataset.

### Assigning Read Pairs to Types

Each read pair is then classified into one of the following categories:

(1) Overlapping 5′ Deletion Breakpoint and Supporting Reference:

A read pair where one or both sequences span at least a predetermined continuous sequence (e.g., 8 bp) on either side of the 5′ deletion breakpoint and both reads are mapped within a predetermined length (e.g., 2 kb base pairs) of the junction.

(2) Overlapping 3′ Deletion Breakpoint and Supporting Reference:

A read pair where one or both sequences span at least a predetermined continuous sequence (e.g., 8 bp) on either side of the 3′ deletion breakpoint and both read pairs are mapped within a predetermined length (e.g., 2 kb base pairs) of the junction.

(3) Supporting Deletion Haplotype:

A paired read that meets one or more of the following conditions:

### (a) Sequences Across Deletion Breakpoints:

If the read aligns to the sequence created by the deletion, covering at least a predetermined continuous sequence (e.g., 8 bp) on either side of the junction formed by the deletion. Candidate reads for this criterion are identified by examining the deletion start and end points and looking for reads with a predetermined range (e.g., 8 or more) soft clipped bases around that position. Reads meeting this criterion are completely realigned to the expected deletion sequence, e.g., by the Smith-Waterman algorithm, to check for overlap and verify that they have the expected sequence.

### (b) Pairs on Opposite Sides:

If the read has paired ends that begin on opposite sides of the deletion, even if the junction sequence is not contained in the reads.

(4) Pairs Contained within the Deleted Region:

Read pairs whose start and end alignments are enclosed within the deleted region.

(5) Pairs not Near Deletion:

Read pairs aligning upstream or downstream of the junction formed by the deletion that provide no information.

(6) Uncertain Pairs:

A read pair where one read is unmapped or the reads do not meet any of the criteria for the other categories (for example a soft clipped read at the deletion junction but with <8 bases on one side of it).

Embodiments may tally up one or more of these types of read pairs (e.g., present in the dataset) and may display them to the user. If any read pair represents the deletion haplotype (Type #3), the program may report that the associated sample or subject is a carrier.

### Sequence Data

To establish a conservative criterion that ensures enough data is present to detect the deletion haplotype in a sample, the program examines the ratio of reads that sequenced either the expected reference sequence at the 3′ breakpoint (Type #2) or the expected deletion haplotype sequence (Type #3a). This ratio may be similar across samples and used to determine how many reads representing the reference sequence would need to be detected to be confident that the haplotype is deleted in an individual. In two known heterozygous samples, the percentage of reads that came from the deletion haplotype was 38% and 37%, respectively (Table 1). To be conservative, a lower ratio of 30% was assumed and the binomial cumulative density function was utilized to determine how many reads are required so that 99.9% of all replicate sets would contain two reads from the deletion if it was present and all samples had this minimum coverage level. This gave a decision criterion of using 35 read pairs, such that, a process or processor will return an error if less than this number of pairs sequencing the reference sequence at the 3′ junction is present. This criterion may be considered conservative because it does not consider the additional evidence provided from pairs that span the deletion junction but do not sequence it.

TABLE 1
Count of read pairs supporting the deletion and specific reads that
overlapped and contained 8 bp of sequence data reading through
the 3′ breakpoint of the deletion (within exon 7) from known
heterozygous individuals supporting each haplotype.

| | | | w/3′ | |
|---|---|---|---|---|
| | Supporting | w/Deletion | Reference | Deletion % of |
| Subject | Deletion | Sequence | Sequence | Total (3′) |
| 67098 | 63 | 48 | 78 | 38% |
| NA02533 | 150 | 132 | 228 | 37% |

### Method Validation

The analysis was run on two positive controls and 123 negative controls. For positive controls, one sample was used that previously was identified to have the mutation by a PCR method as well as a known heterozygous sample available from Coriell, NA02533. For negative controls, 123 client samples were used (All “C” samples to date), that had been previously tested by PCR for the MCOLN1 deletion haplotype. All example samples were sequenced on Illumina machines after enrichment with either the TruSight One Sequencing panel or a custom panel composed of mixing TruSight One and TruSight Inherited Disease panels together.

Both positive samples were readily identified as carriers with a large number of reads sequencing the deletion (Table 1). Of the 123 negative controls, 117 (95%) were identified as negative, while the remainder (only 5 samples) did not meet the minimum coverage of 35 read pairs (having between 14 and 34 read pairs, FIG. 12).

### Program Interface

A program operating according to embodiments may run on any platform. The program may be invoked by a simple command, which inputs the name of the BAM file to analyze and an output file to place a tab delimited file of results. In addition to writing to the output file, the program may print the analysis result and a summary of supporting evidence to the standard output pipe (stdout). FIG. 14 shows an example command and output when run on a known carrier.

### Hardware and System Implementation

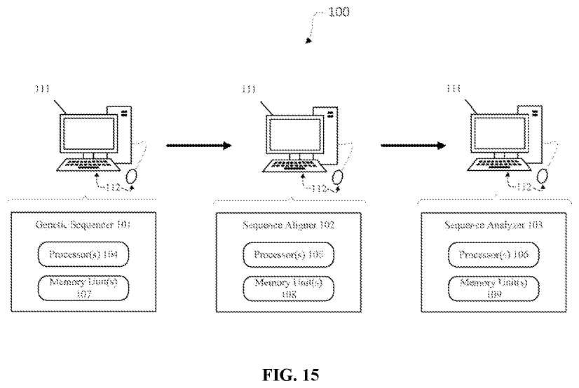

FIG. 15 schematically illustrates a system 100 for sequencing, aligning, and/or analyzing one or more genomes to identify copy number variants (CNVs) for a genetic disease and/or analyzing an exome of one or more genomes, according to an embodiment. In some embodiments, the CNVs are in one or more exons of a gene of interest located on a chromosome, including but not limited to the X chromosome. In some embodiments, system 100 may include a genetic sequencer 101, a sequence aligner 102 and/or a sequence analyzer 103. In some embodiments, the analysis may be used for performing an improved detection of a relatively large predefined deletion in a reference founder genome using short read exome sequencing, according to an embodiment. In one example, the relatively large predefined deletions in a reference founder genome comprise a nearly 6.5 kb deletion in MCOLN1. In some embodiments, system 100 may include a genetic sequencer 101, a sequence aligner 102 and/or a sequence analyzer 103. Units 101-103 may be implemented in one or more computerized devices as hardware and/or software units, for example, specifying instructions configured to be executed by a processor. One or more of units 101-103 may be implemented as separate devices or combined as an integrated device.

Genetic sequencer 102 may input DNA obtained from biological samples, such as, blood, tissue, or saliva, of one or more real living organisms and may output each organism's genetic sequence including the organism's genetic information at one or more genetic loci, for example, a human genome. A single organism's DNA sample may be sequenced for performing carrier testing on that individual.

Sequence aligner 102 may align, whenever possible, reads of a genetic sequence or patient or subject being screened with specific reference points (a read pair aligning to a sequence created by a deletion covering at least 8 bp on either side of the junction formed by the deletion and/or a read pair having paired ends that begin on opposite sides of the deletion reference points) of a reference genetic sequence. In some embodiments, a sequence aligner need not be used.

Sequence analyzer 103 may input multiple sequence alignments and may compute measures to perform various operations relating to identification of copy number variants (CNVs) for a genetic disease (to predict carrier status for exon-level CNVs of a gene of interest), including CNVs in DMD. Sequence analyzer 103 may read and then incorporate counts for the plurality of exon targets outside of the normal range of the prior distribution model indicative of a CNV carrier status of the genetic disease, wherein when the read counts are above normal, the CNV is a duplication and wherein when the read counts are below normal, the CNV is a deletion the normal range of the prior distribution model; a multinomial distribution; and/or a non-conjugate logistic normal prior distribution, and may perform other functions of embodiments as will be described herein. Sequence analyzer 103 may also input multiple sequence alignments and may compute measures to perform various operations relating to prediction of carrier status for deletion mutations of a gene of interest, such as, for example, an approximately 6.5 kb deletion in MCOLN1, and other functions of embodiments described herein.

Genetic sequencer 101, sequence aligner 102, and sequence analyzer 103 may include one or more controller(s) or processor(s) 104, 105, and 106, respectively, configured for executing operations and one or more memory unit(s) 107, 108, and 109, respectively, configured for storing data such as genetic information or sequences and/or instructions (e.g., software) executable by a processor, for example for carrying out methods as disclosed herein. Processor(s) 104, 105, and 106 may include, for example, a central processing unit (CPU), a digital signal processor (DSP), a microprocessor, a controller, a chip, a microchip, an integrated circuit (IC), or any other suitable multi-purpose or specific processor or controller. Processor(s) 104, 105, and 106 may individually or collectively be configured to carry out embodiments of a method according to the present invention by for example executing software or code. Memory unit(s) 107, 108, and 109 may include, for example, a random access memory (RAM), a dynamic RAM (DRAM), a flash memory, a volatile memory, a non-volatile memory, a cache memory, a buffer, a short term memory unit, a long term memory unit, or other suitable memory units or storage units. Genetic sequencer 101, sequence aligner 102, and/or sequence analyzer 103 may include one or more input/output devices, such as output display 111 (e.g., such as a monitor or screen) for displaying to users results provided by sequence analyzer 103, and an input device 112 (e.g., such as a mouse, keyboard or touchscreen) for example to control the operations of system 100 and/or provide user input or feedback.

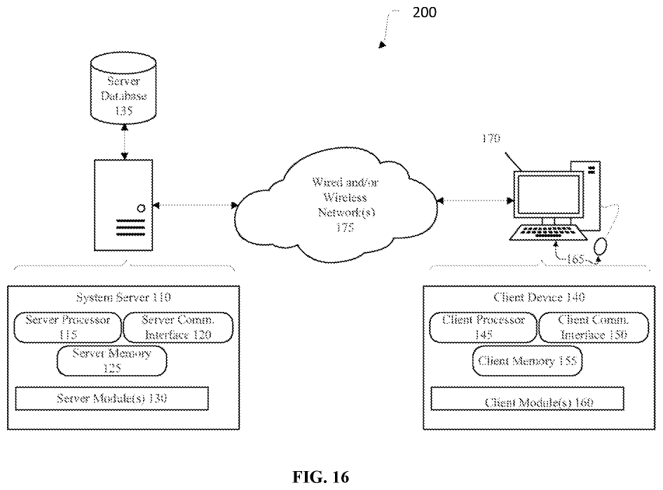

FIG. 16 is a schematic illustration of a system 200 for identifying copy number variants (CNVs) for a genetic disease, according to an embodiment. System 200 may include network 175, which may include the Internet, one or more telephony networks, one or more network segments including local area networks (LAN) and wide area networks (WAN), one or more wireless networks, or a combination thereof. System 200 also includes a system server 110 constructed in accordance with one or more embodiments. In some embodiments, system server 110 may be a stand-alone computer system. In other embodiments, system server 110 may include a network of operatively connected computing devices, which communicate over network 175. Therefore, system server 110 may include multiple other processing machines such as computers, and more specifically, stationary devices, mobile devices, terminals, and/or computer servers (collectively, “computing devices”). Communication with these computing devices may be, for example, direct or indirect through further machines that are accessible to the network 175.

System server 110 may be any suitable computing device and/or data processing apparatus capable of communicating with computing devices, other remote devices or computing networks, receiving, transmitting and storing electronic information and processing requests as further described herein. System server 110 is therefore intended to represent various forms of digital computers, such as laptops, desktops, workstations, personal digital assistants, servers, blade servers, mainframes, and other appropriate computers and/or networked or cloud based computing systems capable of employing the systems and methods described herein.

System server 110 may include a server processor 115 which is operatively connected to various hardware and software components that serve to enable operation of the system 200. Server processor 115 may be configured to execute instructions or software to perform various operations relating to an identification of copy number variants (CNVs) for a genetic disease, e.g., CNVs in DMD, as well as other functions of embodiments. Server processor 115 may also be configured to execute instructions or software to perform various operations relating to prediction of carrier status (e.g., heterozygous) of a large deletion haplotype (e.g., in MCOLN1) in a reference founder genome and/or associated genetic diseases, as well as other functions of embodiments. Server processor 115 may be one or multiple processors, such as a central processing unit (CPU), a graphics processing unit (GPU), a multi-processor core, or any other type of processor, depending on the particular implementation.

System server 110 may be configured to communicate via server communication interface 120 with various other devices connected to network 175. For example, server communication interface 120 may include but is not limited to, a modem, a Network Interface Card (NIC), an integrated network interface, a radio frequency transmitter/receiver (e.g., Bluetooth wireless connection, cellular, Near-Field Communication (NFC) protocol, a satellite communication transmitter/receiver, an infrared port, a USB connection, and/or any other such interfaces for connecting the system server 110 to other computing devices and/or communication networks such as private networks and the Internet.

In some embodiments, a server memory 125 is accessible by server processor 115, thereby enabling server processor 115 to receive and execute instructions such as code, stored in the memory and/or storage in the form of one or more software modules 130, each software module representing one or more code sets or software. The software modules 130 may include one or more software programs or applications (collectively referred to as the “server application”) having computer program code or a set of instructions executed partially or entirely in or by server processor 115 for carrying out operations for aspects of the systems and methods described herein, and may be written in any combination of one or more programming languages. Server processor 115 may be configured to carry out embodiments of the present invention by for example executing code or software, and may be or may execute the functionality of the modules as described herein. [0040] In accordance with various embodiments, server modules 130 may be executed entirely on system server 110 as a stand-alone software package, partly on system server 110 and partly on a client device 140, or entirely on client device 140.

Server memory 125 may be, for example, a random access memory (RAM) or any other suitable volatile or non-volatile computer readable storage medium. Server memory 120 may also include storage which may take various forms, depending on the particular implementation. For example, the storage may contain one or more components or devices such as a hard drive, a flash memory, a rewritable optical disk, a rewritable magnetic tape, or some combination of the above. In addition, the memory and/or storage may be fixed or removable. In addition, memory and/or storage may be local to the system server 110 or located remotely.

In accordance with some embodiments, system server 110 may be connected to one or more database(s) 135, for example, directly or remotely via network 175. Database 135 may include any of the memory conFIGurations as described above, and/or may be in direct or indirect communication with system server 110.

Among the computing devices on or connected to the network 175 may be one or more client devices 140. Client device 140 may be any standard computing device. As understood herein, in accordance with one or more embodiments, a computing device may be a stationary computing device, such as a desktop computer, kiosk and/or other machine, each of which generally has one or more processors, such as client processor 145, configured to execute code or software to implement a variety of functions, a client communication interface 150, a computer-readable memory, such as client memory 155, for connecting to the network 175, one or more client modules, such as client module(s) 160, one or more input devices, such as input devices 165, and one or more output devices, such as output devices 170. Typical input devices, such as, for example, input devices 165, may include, for example, a keyboard, a pointing device (e.g., mouse or digitized stylus), a web-camera, and/or a touch-sensitive display, etc. Typical output devices, such as, for example, output device 170 may include one or more of a monitor, display, speaker, printer, etc.

In some embodiments, client module 160 may be executed by client processor 145 to provide the various functionalities of client device 140. In particular, in some embodiments, client module 160 may provide a client-side interface with which a user of client device 140 may interact, to, among other things, provide a previously unscreened DNA sample or genetic map for carrier screening, as described herein.

Additionally or alternatively, a computing device may be a mobile electronic device (“MED”), which is generally understood in the art as having hardware components as in the stationary device described above, and being capable of embodying the systems and/or methods described herein. A computing device may further include componentry such as wireless communications circuitry, gyroscopes, inertia detection circuits, geolocation circuitry, touch sensitivity, among other sensors. Non-limiting examples of typical MEDs are smartphones, personal digital assistants, tablet computers, and the like, which may communicate over cellular and/or Wi-Fi networks or using a Bluetooth or other communication protocol. Typical input devices associated with conventional MEDs include, keyboards, microphones, accelerometers, touch screens, light meters, digital cameras, and the input jacks that enable attachment of further devices, etc.

In some embodiments, client device 140 may be a “dummy” terminal, by which processing and computing may be performed on system server 110, and information may then be provided to client device 140 via server communication interface 120 for display and/or basic data manipulation. In some embodiments, modules depicted as existing on and/or executing on one device may additionally or alternatively exist on and/or execute on another device. In some embodiments, one or more components of system 100 may be unnecessary to perform aspects of the invention. For example, in embodiment in which NGS data is provided, e.g., by a third party or directly by a subject, the need for genetic sequencer 101 would be obviated.

Embodiments may include an article such as a non-transitory computer or processor readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein.

In some embodiments, provided herein are systems for detecting relatively large predefined deletions in a reference founder genome using short read exome sequencing comprising: a computer having: a processor; a memory storing a target sequence of a reference founder genome that has predefined deletion(s) having a length of base pairs that is relatively larger than a threshold value; and one or more code sets stored in the memory and executing in the processor, which, when executed, configure the processor to: for a plurality of short read exome sequences of continuous exomes segments of a reference genome each having a length of base pairs that is less than or equal to the threshold value (e.g., 150 base pairs); aligning a plurality of short read exome sequences of a sample genetic sequence from a subject to a plurality of short read exome sequences of continuous exomes segments of a reference genome; tallying each aligned read pair; classifying the tallied read pair as at least one of: (a) an aligned sequence comprising a segment positioned after the deletion is shifted to abut a segment positioned prior to the deletion; and (b) an aligned short read pairs comprising paired ends, the paired ends comprising a first nucleic acid sequence read from one end of the target sequence of the reference founder genome and a second nucleic acid sequence read from an opposite end of the target sequence of the reference founder genome; wherein a classification of at least (a) or (b) represents a deletion haplotype; displaying the classified read pair to a user; and reporting the sample genetic sequence as a carrier when the read pair is classified as at least (a) or (b).

In some embodiments of the system, the system is further configured to verify the presence of a minimum threshold (e.g., 35) of short read pairs of exome sequences of the sample genetic sequence from the subject, e.g., to report the sample genetic sequence as a carrier negative wherein if a classified read pair is not at least (a) or (b).

In some embodiments of the system, the system is further configured to determine whether each of the segment before the deletion and the segment positioned prior to the deletion comprise at least a predetermined number (e.g., 8) of base pairs on either side of a junction formed by the deletion.

The descriptions, examples, methods and materials presented in the claims and the specification are not to be construed as limiting but rather as illustrative only. While certain features of the present invention have been illustrated and described herein, many modifications, substitutions, changes, and equivalents may occur to those of ordinary skill in the art. It is, therefore, to be understood that the appended claims are intended to cover all such modifications and changes as fall with the true spirit of the invention.

While the invention has been described with respect to a limited number of embodiments, these should not be construed as limitations on the scope of the invention, but rather as exemplifications of some of the preferred embodiments. Other possible variations, modifications, and applications are also within the scope of the invention. Different embodiments are disclosed herein. Features of certain embodiments may be combined with features of other embodiments; thus certain embodiments may be combinations of features of multiple embodiments.

In addition to the embodiments specifically described above, those of skill in the art will appreciate that the invention may additionally be practiced in other embodiments. Within this written description, the particular naming of the components, capitalization of terms, the attributes, data structures, or any other programming or structural aspect is not mandatory or significant unless otherwise noted, and the mechanisms that implement the described invention or its features may have different names, formats, or protocols. Further, the system may be implemented via a combination of hardware and software, as described, or entirely in hardware elements. Also, the particular division of functionality between the various system components described here is not mandatory; functions performed by a single module or system component may instead be performed by multiple components, and functions performed by multiple components may instead be performed by a single component. Likewise, the order in which method steps are performed is not mandatory unless otherwise noted or logically required. It should be noted that the process steps and instructions of the present invention could be embodied in software, firmware or hardware, and when embodied in software, could be downloaded to reside on and be operated from different platforms used by real time network operating systems.

Algorithmic descriptions and representations included in this description are understood to be implemented by computer programs. Furthermore, it has also proven convenient at times, to refer to these arrangements of operations as modules or code devices, without loss of generality.

Unless otherwise indicated, discussions utilizing terms such as “selecting” or “computing” or “determining” or the like refer to the action and processes of a computer system, or similar electronic computing device, that manipulates and transforms data represented as physical (electronic) quantities within the computer system memories or registers or other such information storage, transmission or display devices.

The algorithms and displays presented are not inherently related to any particular computer or other apparatus. Various general-purpose systems may also be used with programs in accordance with the teachings above, or it may prove convenient to construct more specialized apparatus to perform the required method steps. The required structure for a variety of these systems will appear from the description above. In addition, a variety of programming languages may be used to implement the teachings above.

Finally, it should be noted that the language used in the specification has been principally selected for readability and instructional purposes, and may not have been selected to delineate or circumscribe the inventive subject matter. Accordingly, the disclosure of the present invention is intended to be illustrative, but not limiting, of the scope of the invention.

1. A method for detecting a deletion in a DNA sample using short read exome sequencing, the method comprising:
obtaining short read exome sequences of continuous exome segments of the DNA sample, each exome segments having a length of base pairs that is less than or equal to a threshold value;
receiving a reference sequence of the reference genome, the reference sequencing having a length of base pairs that is larger than the threshold value, the reference sequencing comprising a sequence representing the deletion, a segment positioned prior to the deletion, and a segment positioned after the deletion; and
detecting instances of short read exome sequences that straddle both the segment positioned after the deletion and the segment positioned prior to the deletion, wherein both segments falling within the length of the short read exome sequences indicates that the sequence of the deletion has been deleted in the DNA sample.

2. The method of claim 1, wherein the obtained short read exome sequences are a plurality of short read pairs of exome sequencing data from the DNA sample, the short read pairs comprising paired ends, the paired end comprising a first nucleic acid sequence read from one end of the reference sequence of the reference genome and a second nucleic acid sequence read from an opposite end of the reference sequence of the reference genome.

3. The method of claim 2, wherein each of the first nucleic acid sequence read and the second nucleic acid sequence read is on an opposite side of a deletion junction of the deletion, in a known positional relationship in the reference genome, wherein the reference genome comprises a wild type nucleic acid sequence without any predefined deletions.

4. The method of claim 2, wherein each of the first nucleic acid sequence read and the second nucleic acid sequence read comprises 150 nucleic acid base pairs.

5. The method of claim 1, wherein the reference sequence of the reference genome comprises a nucleic acid sequence in an exome of a gene of interest.

6. The method of claim 3, wherein the nucleic acid sequence spans a 3′ breakpoint position in the gene of interest.

7. The method of claim 1, further comprising aligning nucleic acid sequences of the plurality of short read pairs of exome sequencing data with the reference sequence of the reference genome to obtain a matched alignment of short read pairs of exome sequencing data to the stored reference sequence of the reference genome.

8. The method of claim 1, further comprising visualizing the matched alignment of short read pairs of exome sequencing data to the stored reference sequence of the reference genome.

9. The method of claim 8, wherein the matched alignment of the short read pairs of exome sequencing data comprises an aligned first nucleic acid sequence read and an aligned second nucleic acid sequence read, each nucleic acid sequence read begins on either side of the deletion junction and each of the first and second nucleic acid sequence read does not comprise a deletion junction sequence.

10. The method of claim 9, further comprising realigning the aligned first nucleic acid sequence read and the aligned second nucleic acid sequence read to an expected nucleic acid deletion sequence for the gene of interest, wherein a matched realignment to the expected nucleic acid deletion sequence confirms the subject is a heterozygous carrier of the large base pair deletion.

11. (canceled)

12. (canceled)

13. The method of claim 1, wherein a causative mutation of the relatively large predefined deletion in the reference genome is an insertion or deletion (INDEL) of nucleic acid bases in a gene of the reference genome.

14-16. (canceled)

17. The method of claim 7, wherein absence of a matched alignment of short read pairs of exome sequencing data comprising at least 8 base pairs on either side of the deletion junction is required in a minimum of 35 short read pairs to determine deletion is not present in the DNA sample.

18-21. (canceled)

22. A method for detecting a relatively large predefined deletion in a reference genome using short read exome sequencing, performed on a computer having a processor, memory, and one or more code sets stored in the memory and executing in the processor, the method comprising:
for a plurality of short read exome sequences of continuous exomes segments of a reference genome each having a length of base pairs that is less than or equal to the threshold value;
aligning a plurality of short read exome sequences of a sample genetic sequence from a subject to the reference genome;
tallying each aligned read pair;
classifying the tallied read pair as at least one of:
(a) an aligned sequence comprising a segment positioned after the deletion is shifted to abut a segment positioned prior to the deletion; and
(b) an aligned short read pair comprising paired ends, the paired ends comprising a first nucleic acid sequence read from one end of the reference sequence of the reference genome and a second nucleic acid sequence read from an opposite end of the reference sequence of the reference genome, wherein a classification of at least (a) or (b) represents a deletion haplotype;
displaying the classified read pair to a user; and
reporting the sample genetic sequence as a carrier when the read pair is classified as at least (a) or (b).

23. The method of claim 22, further comprises verifying presence of a minimum of 35 short read pairs of exome sequences of the sample genetic sequence from the subject to report the sample genetic sequence as a carrier negative wherein if a classified read pair is not at least (a) or (b).

24. The method of claim 22, further determining whether each of the segment before the deletion and the segment positioned prior to the deletion comprise at least 8 base pairs on either side of a junction formed by the deletion.

25. The method of claim 22, wherein the threshold value is 150 base pairs.

26. A method of identifying copy number variants (CNVs) for a genetic disease, the method comprising:
generating a prior distribution model defining a normal range of proportional read counts for each of a plurality of exons in one or more genes based on a sample set of training genomes sequenced from DNA of subjects not expressing the genetic disease, the prior distribution model comprising a multi-variate logistic normal model in which the normal range of proportional read counts for each exon is specified by its marginal distribution in a random vector;
receiving a plurality of read counts for exon targets sequenced from DNA of a subject undergoing screening for a genetic disease; and
determining if the subject has read counts for the plurality of exon targets outside of the normal range of the prior distribution model indicative of a carrier status of the genetic disease, wherein when the read counts are above normal, the CNV is a duplication and wherein when the read counts are below normal, the CNV is a deletion.

27. The method of claim 26, wherein a mean vector and covariance matrix determine normal ranges for the normalized counts of the target exons across multiple dimensions of the model.

28. The method of claim 26, further comprising incorporating a non-conjugate logistic normal prior distribution.

29. The method of claim 26, wherein the identified CNVs are in one or more exon.

30-37. (canceled)
